(19) United States
(12) Patent Application Publication
(10) Pub. No.: US 2021/0299452 A1
(43) Pub. Date: Sep. 30, 2021

(54) METHOD AND DEVICE FOR NEURO-STIMULATION

(71) Applicant: SCENERAY CO., LTD., Jiangsu (CN)

(72) Inventors: Weiran Zhu, Jiangsu (CN); Lei Chen, Jiangsu (CN); Yihua Ning, Jiangsu (CN)

(73) Assignee: SCENERAY CO., LTD., Jiangsu (CN)

(21) Appl. No.: 17/215,632

(22) Filed: Mar. 29, 2021

(30) Foreign Application Priority Data
Mar. 30, 2020 (CN) 202010236458.2

(51) Int. Cl. A61N 1/36; A61N 1/05; A61N 1/02; G16H 20/30; G16H 20/70; G06N 20/00

(52) U.S. Cl. CPC A61N 1/36139; A61N 1/0534; A61N 1/0531; A61N 1/36089; G06N 20/00; A61N 1/025; G16H 20/30; G16H 20/70; A61N 1/36096

(57) ABSTRACT

Provided are a method and device for neuro-stimulation. The method for neuro-stimulation includes: detecting a physiological activity signal of each brain region in at least one brain region of a patient through an implantable electrode device; comparing the detected physiological activity signal of the each brain region with a preset detection condition to determine whether the detected physiological activity signal of the each brain region belongs to an abnormal physiological activity signal; and controlling whether to apply an electrical stimulation signal to the at least one brain region through the implantable electrode device based on a determination result of the physiological activity signal of the each brain region.

### CROSS-REFERENCE TO RELATED APPLICATION(S)

This application claims priority to Chinese Patent Application No. 202010236458.2 filed with the CNIPA on Mar. 30, 2020, the disclosure of which is incorporated herein by reference in its entirety.

### TECHNICAL FIELD

The present application relates to a method and device for neuro-stimulation.

### BACKGROUND

Implantable medical devices, such as cardiac pacemakers, defibrillators, nerve stimulators (deep brain stimulators and peripheral nerve stimulators), drug pumps, and the like, have been widely used for the diagnosis, monitoring, and treatment of diseases. In use, an electronic circuit and a battery element disposed in a closed housing are connected to a sensor and a probe outside the closed housing to monitor a specific part of the body or provide electrical/optical/chemical stimulation.

In the case of a nerve stimulator, for example, the pulse generator disposed in the closed housing is connected to the stimulation electrode through an extended wire so that the pulses generated by the pulse generator are transmitted to the stimulation electrode implanted in a designated location, thereby achieving the electrical stimulation intervention in this location.

Deep brain stimulators are one type of neuro-stimulators, which treat diseases by electrically stimulating the brain of a patient. Some deep brain stimulators have been used in clinical applications, such as for the treatment of Parkinson's syndrome, drug addiction, or other diseases. However, deep brain stimulators typically need to continuously apply electrical stimulation signals to the stimulated site in a brain region. But for non-persistent neurological or psychiatric disorders, the continuous application of electrical stimulation signals to the stimulated site may have a negative impact on the patient, and the continuous application of electrical stimulation signals may result in excessive energy release, resulting in the short lifetime of the deep brain stimulator which makes it difficult for the deep brain stimulator to meet the needs of long-term disease treatment.

Therefore, there is a need to provide an improved device for neuro-stimulation.

### SUMMARY

The present application provides a method for neuro-stimulation that enables targeted neuro-stimulation of a patient by detecting abnormal physiological activity signals of the patient.

In one aspect of the present application, a method for neuro-stimulation is provided. The method includes: detecting a physiological activity signal of each brain region in at least one brain region of a patient through an implantable electrode device; comparing the detected physiological activity signal of each brain region with a preset detection condition to determine whether the detected physiological activity signal of each brain region belongs to an abnormal physiological activity signal; and controlling whether to apply an electrical stimulation signal to the at least one brain region through the implantable electrode device based on a determination result of the physiological activity signal of each brain region.

In another aspect of the present application, a device for neuro-stimulation is provided. The device includes: an implantable electrode device, which is configured to detect a physiological activity signal of each brain region in at least one brain region of a patient; and a controller, which is connected to the implantable electrode device and configured to acquire the physiological activity signal of each brain region detected by the implantable electrode device, compare the physiological activity signal of each brain region with a preset detection condition to determine whether the detected physiological activity signal of each brain region belongs to an abnormal physiological activity signal, and control whether to apply an electrical stimulation signal to the at least one brain region through the implantable electrode device based on a determination result of the physiological activity signal of each brain region.

In another aspect of the present application, a device for neuro-stimulation is further provided. The device includes a non-transitory storage medium, a processor, and an implantable electrode device, where the non-transitory storage medium has an instruction executable by the processor, and the instruction is executed to perform the following steps: detecting a physiological activity signal of each brain region in at least one brain region of a patient through an implantable electrode device; comparing the detected physiological activity signal of each brain region with a preset detection condition to determine whether the detected physiological activity signal of each brain region belongs to an abnormal physiological activity signal; and controlling whether to apply an electrical stimulation signal to the at least one brain region through the implantable electrode device based on a determination result of the physiological activity signal of each brain region.

In another aspect of the present application, a computer-readable storage medium is further provided. The computer-readable storage medium stores a computer program, where a processor executes the program to perform the method for neuro-stimulation provided by any embodiment of the present application.

The above is an overview of the present application, and there may be details that are simplified, generalized or omitted. Therefore, it will be appreciated by those skilled in the art that this section is illustrative only and is not intended to limit the scope of the present application in any way. This overview section is not intended to identify key features or essential features of the claimed subject matter, nor is it intended for use in limiting the scope of the claimed subject matter.

### BRIEF DESCRIPTION OF DRAWINGS

The above-mentioned features and other features of the present application will be understood through the following description and appended claims in conjunction with the drawings. It is to be understood that these drawings depict only a number of implementations of the content of the present application and are therefore not to be construed as limiting the scope of the content of the present application. The content of the present application will be described with reference to the drawings.

FIG. 1 illustrates a structural diagram of a device for neuro-stimulation according to an embodiment of the present application;

FIG. 2 illustrates a structural diagram of an implantable electrode according to an embodiment of the present application;

FIG. 3 illustrates a flowchart of updating an abnormal state mode according to an embodiment of the present application;

FIG. 4 illustrates a flowchart of updating an abnormal state mode according to another embodiment of the present application;

FIG. 5 illustrates a schematic diagram of an example manner of applying an electrical stimulation signal according to an embodiment of the present application;

FIG. 6 illustrates a schematic diagram of another example manner of applying an electrical stimulation signal according to an embodiment of the present application;

FIG. 7 illustrates a flowchart of electrical stimulation parameter adjustment according to an embodiment of the present application;

FIG. 8 illustrates a flowchart of a method for neuro-stimulation according to an embodiment of the present application; and

FIG. 9 illustrates a structural diagram of a device for neuro-stimulation according to another embodiment of the present application.

### DETAILED DESCRIPTION

The following description, reference is made to the drawings which form a part hereof. In the drawings, like symbols generally denote like components unless the context indicates otherwise. The illustrative implementations described in the following description, drawings, and claims are not intended to be limiting.

The inventors of the present application have found that deep brain stimulators generally need to continuously apply electrical stimulation signals to the stimulated site in a brain region, which is reasonable for persistent diseases such as Parkinson's syndrome. However, many other mental and behavioral disorders such as depressive disorders, obsessive compulsive disorders, drug-addictive disorders (for example, relapse prevention after heroin withdrawal), or anorexia, generally do not occur persistently, but intermittently. During the non-episode of such diseases, the deep brain stimulator does not need to apply the electrical stimulation signal to the stimulated site. If electrical stimulation signals are continuously applied to the brain during the non-episode of such diseases, these electrical stimulation signals, particularly electrical stimulation signals at high energy levels applied to some of these diseases, may have a negative impact on the patient. Furthermore, the continuous application of electrical stimulation signals results in excessive energy release, which significantly shortens the lifetime of the deep brain stimulator.

The inventors have also found that, for the above-mentioned intermittent mental and behavioral disorders, abnormalities may occur in electroencephalogram signals in some regions of the brain during the episode of the diseases, for example, the amplitude of the electroencephalogram signals may be significantly increased compared to the amplitude of the brain electrical signal during the non-episode period. Such abnormal changes or states of the electroencephalogram signals may serve as a condition for monitoring whether the disease is attacking. Therefore, if such abnormal electroencephalogram signals can be detected, the electrical stimulation signal is applied to the brain region in the event that the abnormal electroencephalogram signals are detected, and thus the deep brain nerve stimulator does not need to continuously apply the electrical stimulation signal to the stimulated site in the brain region.

In the present application, the device for monitoring physiological activities of a region of a patient's brain is integrated into the deep brain stimulator, and the electrical stimulation signal is applied to the patient's brain in a targeted manner by monitoring the abnormal physiological activity of the patient's brain, which greatly improves the efficiency of neuro-stimulation and reduces side effects caused by unnecessary electrical stimulation. In some examples, the method and device of the present application can also generate individualized judgment conditions based on historical data of abnormal physiological activity signals of each patient, thereby enabling the application of electrical stimulation signals to more accurately adapt to individual situations of different patients, which further improves the therapeutic effect.

The method and device of the present application will be described hereinafter with reference to the specific embodiments.

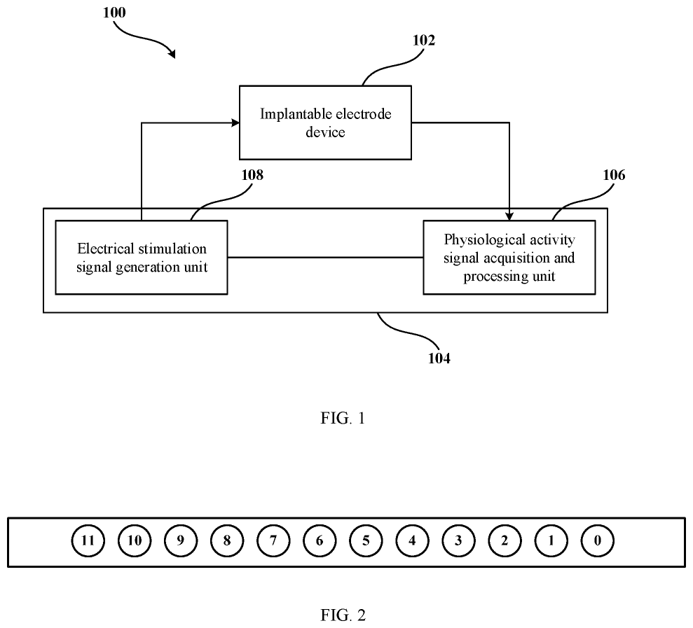

FIG. 1 illustrates a structural diagram of a device 100 for neuro-stimulation according to an embodiment of the present application. The device 100 can perform neuro-stimulation on the brain region of the patient to treat some mental and behavioral disorders such as drug addiction, obsessive compulsive disorders, depressive disorders, and epilepsy, or treat other neurological diseases such as Parkinson's syndrome. It is to be understood that a physician can treat the patient with the device for neuro-stimulation described in the various embodiments of the present application, depending on the need for treatment of a variety of diseases, which is not limited herein. In some embodiments, the method for neuro-stimulation may be configured as an instruction executable by a processor, and the instruction may be stored in a non-transitory storage medium and executed after being run by the processor.

As shown in FIG. 1, the device 100 may include an implantable electrode device 102 and a controller 104. The implantable electrode device 102 may be surgically implanted into the brain of a patient and is configured to detect physiological activity signals of one or more brain regions of the patient and/or apply electrical stimulation signals to one or more brain regions of the patient. In some embodiments, the implantable electrode device 102 may be constructed as a probe or a similar structure, on the outer surface of which one or more electrode contacts may be exposed to contact designated brain regions, respectively. These electrode contacts may be electrically coupled to the interior of the probe via conductive elements to be electrically connected to the controller 104.

In some embodiments, the implantable electrode device 102 may include one implantable electrode, and this implantable electrode may be provided with one or more electrode contacts. In some examples, one or more electrode contacts may sever as both detection contacts and stimulation contacts. For example, one electrode contact may detect and stimulate a brain region at different times, and specifically, the brain region may be detected first and then stimulated by the electrode contact, and then the brain region may be repeatedly detected and stimulated one or more times by the electrode contact. In some other examples, some of the electrode contacts may serve as detection contacts for detecting physiological activity signals of brain regions contacted by the detection contacts, the other electrode contacts may serve as stimulation contacts for applying electrical stimulation signal to brain regions contacted by the stimulation contacts. For example, the implantable electrode may include at least one detection contact for detection and at least one stimulation contact for stimulation, or the implantable electrode may also include multiple detection contacts and multiple stimulation contacts. Optionally, the detection contacts and the stimulation contacts may be configured in pairs or in groups. For example, the number of detection contacts may be the same as the number of stimulation contacts on the implantable electrode, and the location of each detection contact in the brain region substantially overlaps the location of the stimulation contact which is paired with the detection contact in the each brain region. For example, the range of the brain region detected by each detection contact overlaps the range of the brain region affected by the stimulation contact which is paired with the each detection contact. For example, the range of the overlap is greater than a preset value, and the preset value, for example, is a preset ratio of a larger range in the range of the brain region detected by the detection contact and the range of brain regions affected by the stimulation contact. For example, the preset ratio is 80%. For example, one detection contact corresponds to one stimulation contact. In this way, after a detection contact detects the presence of an abnormal physiological activity signal in the brain region in which the detection contact is located, the controller may apply an electrical stimulation signal for therapeutic purposes to the brain region through a stimulation contact corresponding to the detection contact after determining that the detection contact detects the presence of an abnormal physiological activity signal in the brain region in which the detection contact is located. Optionally, the implantable electrode device may include multiple stimulation contacts, and each stimulation contact corresponds to at least one detection contact. The each stimulation contact and the at least one detection contact corresponding to the each stimulation contact constitute a detection-stimulation group, and each detection-stimulation group can independently detect the physiological activity signal and apply the electrical stimulation signal. In some examples, the time when each detection-stimulation group performs electrophysiological signal detection and applies the stimulation signal is not synchronous with the time when at least another detection-stimulation group performs electrophysiological signal detection and applies the stimulation signal. In this way, the application of the electrical stimulation signal may be adjusted according to the location of the electrode contacts on the implantable electrode in the brain region to achieve different therapeutic purposes or meet different therapeutic requirements. In some examples, different detection-stimulation groups perform physiological activity signal detection synchronously, and different detection-stimulation groups perform electrical stimulation signal application synchronously. The detection contacts and the stimulation contacts in the same detection-stimulation group may be in the same brain region or in different brain regions.

In one embodiment, the number of the detection contacts is the same as the number of the stimulation contacts, and the range of a brain region detected by each detection contact does not overlap the range of a brain region affected by a respective stimulation contact corresponding to the each detection contact.

In some embodiments, the implantable electrode device 102 may include multiple groups of implantable electrodes, and each group of implantable electrodes includes one or more implantable electrodes. Optionally, at least one group of implantable electrodes may include both detection contacts and stimulation contacts, and the remaining groups of implantable electrodes may only include stimulation contacts or only include detection contacts. In some embodiments, the implantable electrode device 102 may include multiple groups of implantable electrodes, and a prat of groups of implantable electrodes includes only detection contacts while the other prat of groups of implantable electrodes includes only stimulation contacts.

The number of implantable electrodes included in the implantable electrode device 102, as well as the number and type of electrode contacts included on each implantable electrode, may be flexibly set according to actual application requirements. In some embodiments, the implantable electrode device 102 may include two implantable electrodes, where a first implantable electrode may be implanted in a first brain region of the patient, for example, a left deep brain region, and a second implantable electrode may be implanted in a second brain region of the patient, for example, a right deep brain region. With these two implantable electrodes, the left deep brain region and the right deep brain region may be detected synchronously or asynchronously, or the left deep brain region and the right deep brain region may be subjected to electrical stimulation signals synchronously or asynchronously. Each implantable electrode may be provided with multiple detection contacts and/or stimulation contacts to further detect the physiological activity signal in local portions of a brain region that these electrode contacts actually contact or apply the electrical stimulation signal to the local portions.

According to different actual applications, the implantable electrodes included by the implantable electrode device 102 may be implanted in at least one of: a cerebral cortex region or a deep brain region. In some embodiments, the detection contacts and/or stimulation contacts of the implantable electrode may be implanted in the deep brain region, such as at least one of: nucleus accumbens or anterior limb of internal capsule. The inventors have found that some mental and behavioral disorders, such as drug addiction, can be effectively inhibited by applying electrical stimulation signals to these two deep brain regions, nucleus accumbens or anterior limb of internal capsule. In other embodiments, the stimulation contacts of the implantable electrode may be implanted in other brain regions, which, for example, include, but are not limited to, ventral capsule/ventral striatum, anterior thalamic radiation, medial forebrain bundle, bed nucleus of stria terminalis, subgenual cingulate cortex, inferior thalamic peduncle, amygdala, anterior cingulated cortex, lateral habenula, hippocampus, subthalamic nucleus, or globus pallidus pars interna. In some embodiments, the deep brain region includes a left deep brain region and a right deep brain region. Optionally, implantable electrodes may be implanted into both the left deep brain region and the right deep brain region, and the left deep brain region and the right deep brain region may be electrically stimulated via stimulation contacts on the implantable electrodes.

In one embodiment, the electrical stimulation signal is used for the treatment of mental and behavioral disorders.

In one embodiment, the mental and behavioral disorders include addiction, obsessive compulsive disorders, depressive disorders, anxiety disorders, schizophrenia, anorexia nervosa, Tourette's disorder, or Autism disorder.

In one embodiment, the addiction includes substance addiction or non-substance addiction.

In one embodiment, the substance addiction includes drug addiction, alcohol addiction, nicotine addiction, or caffeine addiction, and the non-substance addiction includes gambling addiction, sexual behavior disorder/addiction, or gaming disorder.

In one embodiment, the drug addiction comprises legal drug addiction or illegal drug addiction; the legal drug addiction comprises hallucinogen addiction, inhalant drug addiction, anesthetic drug addiction, sedative drug addiction, hypnotic drug addiction, anxiolytic drug addiction, or stimulant drug addiction; the illegal drug addiction includes opioid drug addiction, cannabis addiction, methamphetamine addiction, or lysergic acid diethylamide (LSD) addiction.

In one embodiment, since mental and behavioral disorders involve abnormalities in a wider range of neural circuits in the brain, such as abnormalities in the midbrain-cortical loop and abnormalities in the midbrain-cortex loop, when the deep brain stimulator is used for the treatment of mental and behavioral disorders, two or more regions need to be intervened at the same time to produce a better therapeutic effect. In one embodiment, the electrical stimulation signal is applied to at least two brain regions.

In one embodiment, the implantable electrodes may be implanted into at least one brain region of the left brain region and at least one brain region of the right brain region.

In one embodiment, the implantable electrodes may be implanted into at least two brain regions of the left brain region and/or at least two brain regions of the right brain region.

In one embodiment, the cerebral cortex region includes at least one of: prefrontal cortex, orbitofrontal cortex, parietal cortex, or temporal cortex.

In one embodiment, the prefrontal cortex includes at least one of: dorsomedial prefrontal cortex or dorsolateral prefrontal cortex.

In one embodiment, the implantable electrodes may be implanted into ventral capsule/ventral striatum, bed nucleus of stria terminalis, anterior limb of internal capsule, and nucleus accumbens, and the controller, through the implantable electrode device, may apply an electrical stimulation signal with a voltage range of 2.5 V to 8 V, a pulse width range of 120 μs to 210 μs, and a frequency range of 90 Hz to 135 Hz to ventral capsule/ventral striatum, apply an electrical stimulation signal with a voltage range of 3 V to 10.5 V, a pulse width range of 90 μs to 450 μs, and a frequency range of 85 Hz to 135 Hz to the bed nucleus of stria terminalis, apply an electrical stimulation signal with a voltage range of 2 V to 7 V, a pulse width range of 90 μs to 300 μs, and a frequency range of 130 Hz to 185 Hz to anterior limb of internal capsule, and apply an electrical stimulation signal with a voltage range of 4 V to 7 V, a pulse width range of 90 μs to 240 μs, and a frequency range of 100 Hz to 150 Hz to nucleus accumbens. It is confirmed through experiments that the above-mentioned solution had a good therapeutic effect on obsessive compulsive disorders.

In one embodiment, the implantable electrodes may be implanted into anterior limb of internal capsule and cingulated cortex, and the controller, through the implantable electrode device, may apply an electrical stimulation signal with a voltage range of 2.5 V to 6 V, a pulse width range of 90 μs to 300 μs, and a frequency range of 130 Hz to 180 Hz to anterior limb of internal capsule and apply an electrical stimulation signal with a voltage range of 2 V to 10 V, a pulse width range of 90 μs to 300 μs, and a frequency range of 130 Hz to 210 Hz to cingulated cortex. It is confirmed through experiments that the above-mentioned solution had a good therapeutic effect on depressive disorders.

In some embodiments, the implantable electrode device includes one or more detection contacts whose diameter is 0.1 mm to 3 mm, and each detection contact may detect a physiological activity signal with an amplitude range of 5 uV to 12.5 mV and a frequency range of 0.5 Hz to 150 Hz. In some embodiments, the implantable electrode device includes one or more detection contacts whose diameter is 0.1 mm to 0.5 mm, and each detection contact may detect a physiological activity signal with an amplitude range of 5 uV to 10 mV and a frequency range of 0.5 Hz to 30000 Hz. These physiological activity signal roughly correspond to electrocorticogram signals and local electroencephalogram signals in the deep brain region. In some embodiments, the stimulation contacts may have a size and configuration similar to the detection contacts.

FIG. 2 illustrates a structural diagram of an implantable electrode according to an embodiment of the present application. In some examples, the implantable electrode device 102 shown in FIG. 1 may include one or more implantable electrodes shown in FIG. 2.

As shown in FIG. 2, the implantable electrode may be configured in a probe structure having an elongated body, and the electrode contacts may be longitudinally distributed along the elongated body to contact brain regions in different locations at preset intervals. The implantable electrode shown in FIG. 2 includes 12 electrode contacts, and in some other examples, the number of electrode contacts may be different from the number of electrode contacts shown in FIG. 2. Furthermore, the electrode contacts shown in FIG. 2 are arranged in an inline manner on the implantable electrode, and in some other examples, the electrode contacts may be arranged in an annular manner, alternately, or in any other desired manners on the implantable electrode. Furthermore, the implantable electrode may be configured in any other shape, such as a bend architecture, a spiral shape, an annular shape, or other shapes.

It is favorable to set multiple electrical contacts on one implantable electrode. For example, when a part of these electrode contacts serves as detection contacts, physiological activity signals detected by multiple adjacent detection contacts may be acquired in a differential manner. In other words, the physiological activity signal actually used for subsequent judgment may be a signal difference of the physiological activity signal between one detection contact (as a sampling point) and another detection contact (as a reference point). Such a differential signal acquisition helps to reduce the interference of noise signals and to extract useful signals. Furthermore, in some embodiments, the number and locations of the detection contacts as sampling points may be configured, while the number and locations of the detection contacts as reference points may also be configured. In other words, any one of the detection contacts may be selected as a sampling point or a reference point. With continued reference to FIG. 2, for example, adjacent detection contacts 4 to 6 may serve as a group of detection contacts, where the detection contact 5 serves as the sampling point and the detection contacts 4 and 6 serve as the reference point. In this way, these three detection contacts may generate a physiological activity signal which reflects the physiological activity around the brain region in which these three detection contacts are located. For the physiological activity signal, the detection contact 5 is used as the signal input for sampling, the signal after the short circuit of the detection contacts 4 and 6 is used as the reference signal for sampling, and the signal difference between the detection contact 5 and the detection contact 4 or 6 is used as the physiological activity signal. For example, the physiological activity signal A detected by the detection contacts 4 to 6 is obtained through the following formula: A=A₅−1/2(A₄+A₆), where Aᵢ is the physiological activity signal detected by the contact i, and i=4, 5, or 6. Similarly, the reference point may be one detection contact or multiple detection contacts; or the reference point may be a proximity detection contact around the sampling point or another detection contact at a certain distance from the sampling point.

Generally speaking, the electrode contacts implanted into the brain generally need to be in contact with brain tissue or nerve tissue to release the electrical stimulation to the human tissue through the contact interface. Therefore, the material of the electrode contacts needs to be a conductive material that has good biocompatibility and good electrochemical corrosion resistance, such as platinum (Pt), platinum-iridium alloy (PtIr), and the like. The shape of the electrode contacts may be annular, dot-shaped, or sheet-shaped. The shape of the electrode contacts needs to be determined according to the location in which the product plans to be implanted and the use of the product. Furthermore, the size of the electrode contacts may be determined according to the number of nerve cells to be stimulated or detected, ranging from 0.01 mm to 6 mm. For example, the size of the contact for a single or several neurons may be between 0.01 mm and 0.1 mm; the size of the contact for hundreds to tens of thousands of neurons may be between 0.1 mm and 0.5 mm; and the size of the contact for functional nucleus or larger size brain tissue may be between 0.5 mm and 6 mm.

In one embodiment, the diameter of the detection contact only for detection ranges from 5 um to 100 um so that the detection contact may detect a small range, for example, detecting the discharge activity signals of a single or less than 100 neurons, to determine whether the small region detected by the detection contact is abnormal. The contacts with a diameter greater than 100 um may be used for both detection and stimulation.

With reference to FIG. 1 again, the device for neuro-stimulation 100 further includes a controller 104. The controller 104 is configured to acquire the physiological activity signal of each brain region in one or more brain regions where the device for neuro-stimulation 100 is located detected by the implantable electrode device 102, and compare the physiological activity signal of each brain region with a preset detection condition to determine whether the detected physiological activity signal belongs to an abnormal physiological activity signal. The controller 104 is further configured to control whether to apply an electrical stimulation signal to one or more brain regions of the patient through the implantable electrode device 102 based on a determination result.

In some embodiments, for example, in the embodiment shown in FIG. 1, the controller 104 may include a physiological activity signal acquisition and processing unit 106 and an electrical stimulation signal generation unit 108. The physiological activity signal acquisition and processing unit 106 is coupled to the implantable electrode device 102 to receive a physiological activity signal acquired by the implantable electrode device 102 and to compare the physiological activity signal with a preset detection condition to judge whether the physiological activity signal is an abnormal physiological activity signal. In some embodiments, the physiological activity signal acquisition and processing unit 106 may include a signal acquisition module and a signal processing module. The signal acquisition module is configured to receive a physiological activity signal and to convert a weak analog biological voltage and current signal embodied by the physiological activity signal into an analog or digital signal available to the subsequent processing. Optionally, the signal acquisition module converts the weak analog biological voltage and current signal embodied by the physiological activity signal into a digital signal. In one example, the signal acquisition module may include a signal isolation circuit, a signal amplification circuit, a signal filtering circuit, a sample-and-hold circuit, a signal analog-digital conversion circuit, and other circuit elements. The signal isolation circuit is configured to isolate the physiological activity signal of the human body from the direct current circuit of an acquisition circuit to prevent physiological damage to the human body due to the direct current leakage flow generated by the circuit. The signal amplification circuit is provided with a variable amplification gain (for example, automatic gain adjustment or a designated gain adjustment), and the signal amplification circuit is configured to adjust a physiological activity signal of a small magnitude (for example, a level at 10 uV to 100 mV) to a physiological activity signal within a voltage range available for subsequent circuit processing. The filtering circuit is configured to filter out interference signals (for example, power line interference, radiated noise, power supply noise, and the like), extract useful signals that are expected to be used, and improve the signal-to-noise ratio when the signal is processed by a subsequent circuit. The sample-and-hold circuit is configured to ensure that the signal applied to the analog-digital conversion circuit remains unchanged within the period of time when the analog-digital conversion circuit completes the analog-digital conversion once, thereby reducing the effect of signal fluctuations on the accuracy of the conversion result. The analog-digital conversion circuit is configured to convert, through high-speed sampling, the analog signal subjected to amplification and filtering into a digital signal that is capable of being stored and calculated by a program. The signal processing module is configured to perform signal processing on the converted digital signal, such as Fourier transform, filtering, and convolution operation, to identify the abnormal physiological activity signal at the time when the disease is attacking and generate an indication or identification corresponding to the abnormal physiological activity signal. It is to be understood that the above description about the hardware circuit of the physiological activity signal acquisition and processing unit 106 is illustrative and that various configurations, designs, and modifications may be made by those skilled in the art in accordance with the needs of the actual application.

The preset detection condition adopted by the controller 104 or by the physiological activity signal acquisition and processing unit 106 may include, for example, a preset threshold or range of one or more parameters (for example, voltage). If the voltage or other parameters of the physiological activity signal exceeds the preset threshold or range, it is indicated that the physiological activity signal is an abnormal physiological activity signal and that the patient is likely to be in a disease attack state at that time. In some embodiments, the preset detection condition may include an abnormal state mode. The abnormal state mode may include a change in one or more parameters of an abnormal physiological activity signal over time. In some examples, these parameters may be the intensity and/or characteristic frequency of the abnormal physiological activity signal. For example, when the disease is not attacking, the amplitude or intensity of the physiological activity signal of the brain region may be relatively low, and the period of the physiological activity signal of the brain region is also randomly disordered. However, when the disease is attacking, the intensity of the physiologically active signal may become relatively high and may vary periodically in a variation curve. The controller 104 may perform a similarity comparison between the signal intensity change acquired by the implantable electrode device 102 and a known intensity change curve. If the similarity is higher than a judgment reference value (for example, 50%), it is considered that the acquired physiological activity signal conforms to the abnormal state mode and the acquired physiological activity signal is an abnormal physiological activity signal. If the similarity is lower than the judgment reference value, it is considered that the acquired physiological activity signal is a normal physiological activity signal, that is, the disease is not attacking at that time. Optionally, the similarity comparison between the acquired physiological activity signal and the abnormal state mode may be performed based on a particular triggering condition, for example, the similarity comparison is initiated/triggered only after the intensity of the physiological activity signal exceeds a preset threshold, which helps to reduce the unnecessary power consumption occupied by the comparison.

In some embodiments, when the similarity comparison is performed, a physiological activity signal during a window period may be acquired, and the correlation operation is performed on the acquired physiological activity signal and the abnormal physiological activity signal included in the preset abnormal state mode. If there is a maximum value in the result of the correlation operation, it may be considered that the disease is attacking and the stimulation is initiated. If there is no maximum value in the result of the correlation operation, the window is moved backwards to acquire another physiological activity signal, and the correlation operation is performed on the newly acquired physiological activity signal and the abnormal physiological activity signal included in the preset abnormal state mode, that is, the cross-correlation operation is performed. In one example, a physiological activity signal acquired within a period of time may be stored, and the correlation operation is performed on the stored physiological activity signal and a physiological activity signal subsequently acquired. If there is a maximum value in the result of the correlation operation, the stimulation is initiated. If there is no maximum value in the result of the correlation operation, the window is moved backwards and the correlation operation is continued, that is, the autocorrelation operation is performed.

In some embodiments, one or more parameters of the abnormal physiological activity signal included in the abnormal state mode may be associated with one brain region. In other embodiments, one or more parameters of the abnormal physiological activity signal included in the abnormal state mode may also be associated with multiple brain regions, that is, the abnormal state mode includes one or more parameters of abnormal physiological activity signals of multiple brain regions because when the disease is attacking, the physiological activity signals of multiple brain regions may become abnormal at the same time and may be abnormally associated with each other. For example, there may be an abnormal intensity change in the physiological activity signal of anterior limb of internal capsule first, and then a similar abnormal intensity change occurs in the physiological activity signal of nucleus accumbens subsequently (for example, several milliseconds or less later). The controller 104 may simultaneously acquire parameter changes in the physiological activity signals of multiple brain regions and judge whether the disease is attacking.

In some embodiments, the abnormal state mode may be generated based on a preset abnormal state mode. The abnormal state mode may be a statistical model generated in advance based on data collected during disease episodes in other patients. In some embodiments, the abnormal state mode may be generated based on one or more historical abnormal physiological activity signals of the patient (for example, a patient implanted with an implantable electrode device). Optionally, the abnormal state mode may be updated immediately according to the historical abnormal physiological activity signal of the patient, and the updating may be performed by training based on a machine learning algorithm. Various machine learning algorithms, for example, deep neural network, may be used for the training of the abnormal state mode, which is not limited in the present application.

Due to the individual difference of the patient, it may not be accurate enough to judge whether the level of physiological activity signals has reached the level at which the disease is attacking by using uniform criteria. Therefore, the preset abnormal state mode may be updated according to the individual condition of the patient to continuously improve the electroencephalogram signal model in the abnormal state mode (that is, one or more parameters of the abnormal physiological activity signal change over time), that is, the process of self-learning perfection is performed. When the deep brain stimulator is initially implanted into the brain of the patient, the electroencephalogram signal model stored in the controller may be a characteristic value of the disease or an empirical value of the physician. When the controller compares the acquired physiological activity signal with the stored electroencephalogram signal model and judges that the acquired physiological activity signal is a physiological activity signal (abnormal physiological activity signal) generated when the disease is attacking, the controller may store the physiological activity signal generated when the disease is attacking and then combine the electroencephalogram signal model with the physiological activity signal generated when the disease is attacking to obtain an optimized electroencephalogram signal model and update the previously stored electroencephalogram signal model.

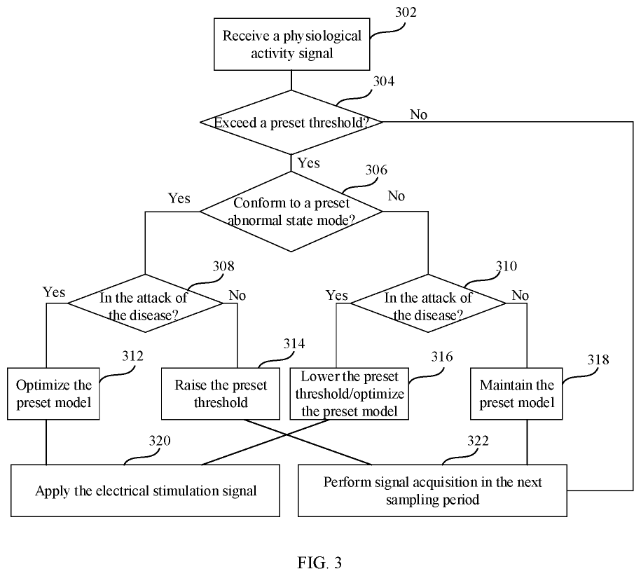

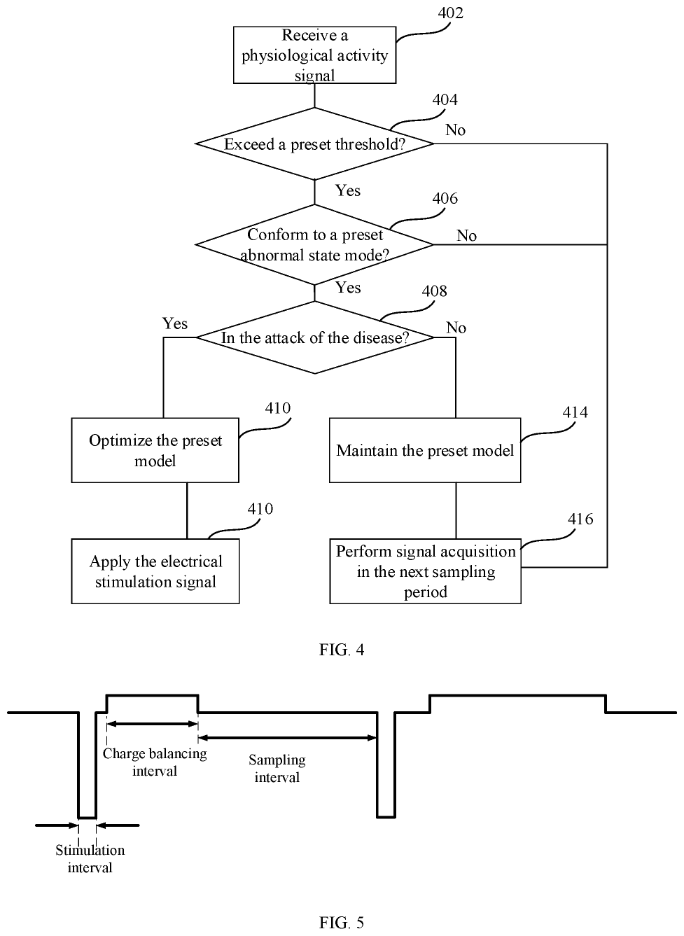

FIGS. 3 and 4 illustrate two example flowcharts of updating an electroencephalogram signal model of an abnormal state mode.

As shown in FIG. 3, the flow begins at step 302. In step 302, the controller receives a physiological activity signal acquired by the implantable electrode device. In step 304, the controller judges whether the physiological activity signal exceeds a preset threshold, that is, the controller determines whether the operation of comparing the physiological activity signal with a preset abnormal state mode is triggered. If the judgment result is that the physiological activity signal does not exceed the preset threshold, go to step 322 to wait for signal acquisition in the next sampling period and then repeat the flow of updating the state mode. If the judgment result in step 304 is that the physiological activity signal exceeds the preset threshold, go to step 306, that is, judge whether the parameter change of the acquired physiological activity signal conforms to the preset abnormal state mode. If the parameter change of the acquired physiological activity signal conforms to the preset abnormal state mode, go to step 308, while if the parameter change of the acquired physiological activity signal does not conform to the preset abnormal state mode, go to step 310. Steps 308 and 310 are both used for judging whether the patient is actually in an attack of the disease. If it is judged in step 308 that the patient is actually in the attack of the disease, which indicates that the judgment result previously obtained in step 306 that the parameter change of the acquired physiological activity signal conforms to the preset abnormal state mode is accurate, in step 312, the physiological activity signal which has been received and is being processed may be stored, and the physiological activity signal which has been received and is being processed may be combined with a preset model associated with the preset abnormal state mode (for example, a mathematical model such as a reinforcement learning model under unconstrained or constrained conditions) to optimize the preset model. If it is judged in step 308 that the patient is not actually in the attack of the disease, which indicates that the judgment result previously obtained in step 306 is inaccurate, a preset threshold may be raised in step 314. Similarly, if it is judged in step 310 that the patient is not actually in the attack of the disease, which indicates that the judgment result previously obtained in step 306 that the parameter change of the acquired physiological activity signal does not conform to the preset abnormal state mode is accurate, the preset model may be maintained unchanged in step 318. If it is judged in step 310 that the patient is actually in the attack of the disease, which indicates that the judgment result previously obtained in step 306 is inaccurate, in step 316, the preset threshold may be lowered and/or the received physiological activity signal may be combined with the preset model to optimize the preset model. In a case where it is judged that the patient is actually in the attack of the disease, go to step 320 to apply an electrical stimulation signal to the patient through the implantable electrode device. In a case where it is judged that the patient is not actually in the attack of the disease, go to step 322 for signal acquisition in the next sampling period.

It is to be understood that through the above-mentioned manner, the abnormal state mode can be updated according to the actual physical condition and episode state of each patient so that the judgment of the abnormal state mode is more accurate, thereby reducing the misoperation and missed operation. The update manner shown in FIG. 3 is relatively complex and thus is suitable for initializing the parameters of the physiological activity signal when the device is just implanted under hospital monitoring.

FIG. 4 illustrates another flowchart of updating an abnormal state mode according to an embodiment of the present application. Compared to the flow of updating shown in FIG. 3, the flow shown in FIG. 4 is relatively simple and is suitable for the patient to update the abnormal state mode when the patient uses the device at home.

As shown in FIG. 4, the flow begins at step 402. In step 402, the controller receives a physiological activity signal acquired by the implantable electrode device. In step 404, the controller judges whether the physiological activity signal exceeds a preset threshold, that is, the controller determines whether the operation of comparing the physiological activity signal with a preset abnormal state mode is triggered. If the judgment result is that the physiological activity signal does not exceed the preset threshold, go to step 416 to wait for signal acquisition in the next sampling period and then repeat the flow of updating the abnormal state mode. If the judgment result in step 404 is that the physiological activity signal exceeds the preset threshold, go to step 406, that is, judge whether the parameter change of the acquired physiological activity signal conforms to the preset abnormal state mode. If the parameter change of the acquired physiological activity signal conforms to the preset abnormal state mode, go to step 408, while if the parameter change of the acquired physiological activity signal does not conform to the preset abnormal state mode, go to step 416. Step 408 is used for judging whether the patient is actually in an attack of the disease. If it is judged in step 408 that the patient is actually in the attack of the disease, which indicates that the judgment result previously obtained in step 406 that the parameter change of the acquired physiological activity signal conforms to the preset abnormal state mode is accurate, in step 410, the physiological activity signal which has been received and is being processed may be stored, and the physiological activity signal which has been received and is being processed may be combined with a preset model (for example, a mathematical model) associated with the preset abnormal state mode to optimize the preset model. If it is judged in step 408 that the patient is not in the attack of the disease, which indicates that the judgment result previously obtained in step 406 is inaccurate, go to step 416 to maintain the preset model unchanged, that is, the current physiological activity signal would not be used for updating. After step 414, go to step 416.

It is to be understood that, when the electroencephalogram signal model of the abnormal state mode does not need to be updated, the controller, after judging whether the preset abnormal state mode is conformed to, may directly apply electrical stimulation according to the judgment result or perform signal acquisition in the next sampling period.

With reference to FIG. 1 again, the controller 104 further includes an electrical stimulation signal generation unit 108. The electrical stimulation signal generation unit 108 is coupled to the physiological activity signal acquisition and processing unit 106 and the implantable electrode device 102. The electrical stimulation signal generation unit 108 is configured to receive a determination result of whether the physiological activity signal generated by the physiological activity signal acquisition and processing unit 106 belongs to an abnormal physiological activity signal and control whether to apply an electrical stimulation signal to one or more brain regions through the implantable electrode device 102 based on the determination result.

In some embodiments, the electrical stimulation signal generation unit 108 may generate an electrical stimulation signal having a therapeutic effect, typically an electrical stimulation pulse, according to a detection or processing result of the physiological activity signal acquisition and processing unit 106. The electrical stimulation signal generation unit 108 may be a current-based stimulation source or may be a voltage-based stimulation source or a charge transfer-based stimulation source. One or more parameters of the electrical stimulation signal may be adjusted. These parameters include, for example, a pulse frequency, a pulse width, a pulse amplitude, a pulse shape (shape of rising edge, falling edge, and pulse base), a duration, and the like.

In some embodiments, the implantable electrode device has multiple stimulation contacts, and the electrical stimulation pulse applied on each stimulation contact may be independent of each other, or may be associated with each other. For example, the electrical stimulation pulse may be in the same frequency and different phases or in the same frequency and the same phase, or the amplitude of the electrical stimulation pulse is incrementing. It is to be understood that the physician may adjust the parameters of the electrical stimulation pulse according to the actual condition of different patients, or adjust the parameters of the electrical stimulation pulse according to the location of a brain region where the stimulation contact is located, or adjust the relationship between these parameters.

In some embodiments, each electrical stimulation may be last for 1 second to 1 hour. In some embodiments, at least one of the duration or amplitude of the electrical stimulation signal may be controlled or adjusted. Optionally, the duration or amplitude of the electrical stimulation signal may be controlled according to the detected physiological activity signal. For example, the duration and amplitude of the applied electrical stimulation signal may be positively correlated with the detected physiological activity signal, that is, the greater the intensity of the physiological activity signal, the longer the duration or the greater the amplitude of the applied electrical stimulation signal.

In some optional embodiments, the electrical stimulation signal generation unit 108 may generate periodic electrical stimulation signals to intermittently electrically stimulate the brain region through the implantable electrode device 102. Similarly, the amplitude, duration, and frequency of the periodic electrical stimulation signal may be positively correlated with the detected physiological activity signal.

In general, the amplitude of the detected physiological activity signal is usually small and may be on the order of uV, while the amplitude of the therapeutic electrical stimulation signal is usually much larger than the order of uV. For example, the amplitude of the therapeutic electrical stimulation signal is between 100 mV and 10 V, optionally between 0.5 V and 10 V. Therefore, if a brain region is stimulated and then the physiological activity signal of this brain region is detected immediately, the sampling circuit would enter the saturated or supersaturated state due to the excessive charge introduced by the electrical stimulation, and thus no valid physiological activity signal can be obtained. To avoid this problem, in some embodiments, the detection of a next physiological activity signal may be performed after a predetermined time interval upon the completion of each application of the electrical stimulation signal. The predetermined time interval may be, for example, 0.01 ms to 1 hour.

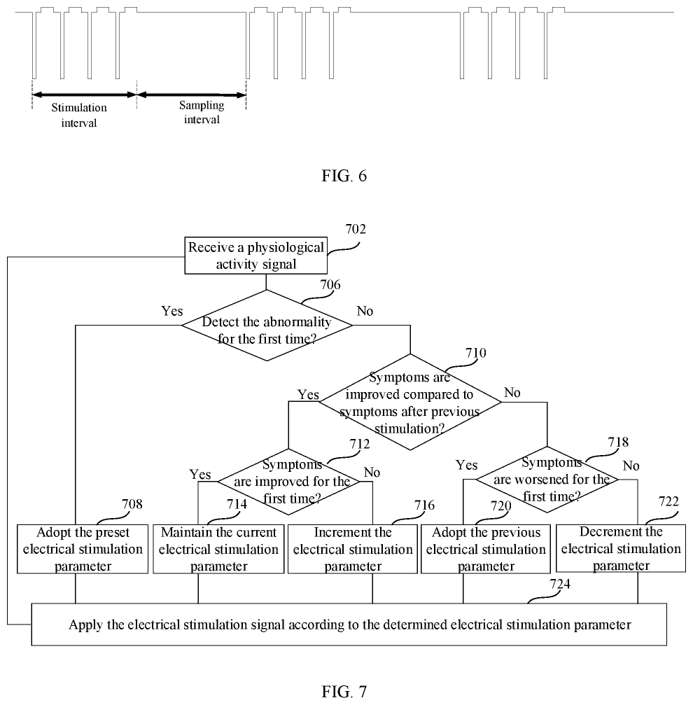

FIGS. 5 and 6 illustrate schematic diagrams of two example manners of applying an electrical stimulation signal. Both manners can avoid the above-mentioned problem of saturation of the sampling circuit.

As shown in FIG. 5, after a period of time upon the completion of the electrical stimulation (for example, electrical stimulation using a pulse signal) of a brain region, there may be a period of time for charge balancing, and then sampling is performed on this brain region after charge balancing. Since the charge balance interval effectively eliminates the excessive charge induced by electrical stimulation, the sampled physiological activity signal can accurately reflect the physiological condition of the brain region. Such a process, including electrical stimulation, charge balancing, and sampling, can be performed periodically to enable to continuously detect and treat the patient.

As shown in FIG. 6, the electrical stimulation and sampling may be performed on the brain region at different times, that is, charge balancing is performed after a period of time upon the completion of electrical stimulation, and then the brain region may be sampled for a period of time. After the completion of sampling of the physiological activity signal, the brain region may continue to be electrically stimulated. The duration of the electrical stimulation operation may be adjusted according to actual requirements, for example, to adjust the number of stimulation pulses.

It is to be understood that as mentioned above, since the electrical stimulation is triggered or initiated based on the occurrence of the abnormal physiological activity signal, the manners of applying the electrical stimulation shown in FIGS. 5 and 6 are performed on the premise that it is determined that there is an abnormal physiological activity signal and the electrical stimulation signal needs to be applied. In some embodiments, the sampling operations shown in FIGS. 5 and 6 are used for determining whether to adjust the parameters of the electrical stimulation signal or whether to stop the application of the electrical stimulation signal (for example, when the patient is not in the attack of the disease).

In the process of applying the electrical stimulation signal, the electrical stimulation signal generation unit may adjust the parameters of the electrical stimulation signal according to the detection result of the physiological activity signal acquisition and processing unit to achieve a better therapeutic effect. In some embodiments, the controller may store parameters of electrical stimulation which has a good therapeutic effect as parameters of electrical stimulation applied for the next time when the disease is attacking.

FIG. 7 illustrates a flowchart of electrical stimulation parameter adjustment according to an embodiment of the present application. In the flowchart, the parameters of electrical stimulation may be dynamically adjusted according to the symptom changes after the patient is applied with the electrical stimulation signal.

As shown in FIG. 7, in step 702, the electrical stimulation signal generation unit receives a physiological activity signal acquired by the physiological activity signal acquisition and processing unit. In step 706, the electrical stimulation signal generation unit determines whether the current reception of the abnormal physiological activity signal is the first time that the abnormal physiological activity signal is received, that is, whether the patient is in the attack of the disease for the first time after being implanted with an implantable electrode. If the patient is in the attack of the disease for the first time, go to step 708. In step 708, a preset electrical stimulation parameter is used, which, for example, is preset by the physician according to the condition of the patient. If it is judged in step 706 that the patient is not in the attack of the disease for the first time, go to step 710. In step 710, it is judged whether the current physiological activity signal (representing current symptoms of the patient in the attack of the disease) is improved compared to the physiological activity signal of the patient after the previous application of the electrical stimulation signal. If the current physiological activity signal is improved compared to the physiological activity signal of the patient after the previous application of the electrical stimulation signal, it is indicated that the effect of the current electrical stimulation parameter is better. In step 712, it is further judged whether the current symptom improvement is the symptom improvement for the first time. If the current symptom improvement is the symptom improvement for the first time, in step 714, the current electrical stimulation parameter is maintained and then used for the next electrical stimulation. If the current symptom improvement is not the symptom improvement for the first time, go to step 716 in which the electrical stimulation parameter is incremented, for example, the electrical stimulation parameter is increased stepwise by a preset value. If it is judged in step 710 that the current symptom is not improved compared to the previous symptom subjected to stimulation (that is, the current symptom becomes worse), go to step 718 to judge whether the current symptom worsening is the symptom worsening for the first time. If the current symptom worsening is the symptom worsening for the first time, the previous electrical stimulation parameter is used for the next electrical stimulation in step 720. If the current symptom worsening is not the symptom worsening for the first time, go to step 722 in which the electrical stimulation parameter is decremented, for example, the electrical stimulation parameter is decreased stepwise by a preset value. After the electrical stimulation parameter is determined in step 708, 714, 716, 720, or 722, in step 724, the electrical stimulation signal generation unit generates an electrical stimulation signal according to the determined electrical stimulation parameter and applies the electrical stimulation signal to the brain region of the patient through the implantable electrode device.

As can be seen, the above-mentioned flow enables the controller to dynamically adjust the electrical stimulation parameter according to the improvement/deterioration of the patient's symptoms. It is to be understood that, in some embodiments, the electrical stimulation signal may have multiple adjustable parameters. Accordingly, one parameter, for example, electrical stimulation voltage amplitude, may be adjusted first, and then one or more other parameters may be adjusted after the competition of the adjustment of this parameter, for example, the duration and frequency of the electrical stimulation voltage.

The controller 104 shown in FIG. 1 may be integrally constructed to be implanted into the body of the patient as a whole or may be constructed in two parts, where one part is implanted into the body of the patient and the other part is disposed outside the patient, for example, the other part is configured as a portable structure. The part of the controller 104 implanted into the body of the patient may be powered by batteries integrated with this part, while the part disposed outside the body may be powered by another battery, and the two parts may be coupled to each other by wireless communication. Optionally, for example, a circuit or module that needs to perform the large data volume calculation may be disposed outside the body and powered by a separate battery, which helps to reduce the power consumption of the battery in the body, thereby prolonging the service life. In some embodiments, the controller 104 may also be coupled to an external device, for example, the controller 104 is coupled to a server or a computer used by the physician by wireless communication and may send the detected physiological activity signal or an electrical stimulation history record to the external device, thereby enabling the physician to monitor the treatment condition of the patient in real time.

As can be seen, the device shown in FIG. 1 can continuously monitor the physiological activity signal of the region where the implantable electrode is located, judge in real time whether the signal level reaches the degree at which the disease is attacking, and once the signal level reaches or exceeds the clinically confirmed disease attack mode, and apply the corresponding level of the electrical stimulation signal to the brain region (the energy level of electrical stimulation is determined by the physician according to the clinical curative effect of the patient). When it is detected that the physiological activity signal does not reach the degree of disease attack, the electrical stimulation signal may be stopped to apply, and the device returns the physiological activity signal detection state. In this way, the side effects of stimulation on the patient who is not in the attack of the disease are reduced, power consumption is reduced, and the service life is prolonged.

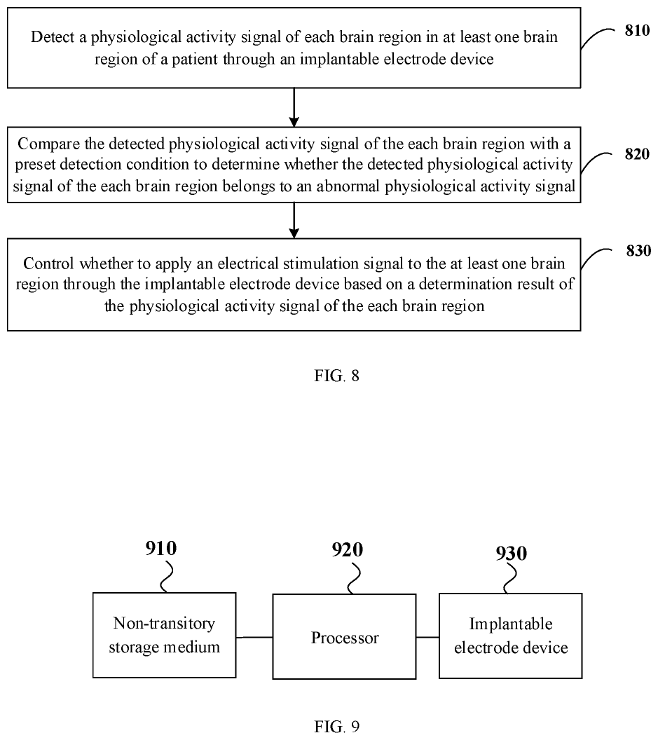

FIG. 8 illustrates a flowchart of a method for neuro-stimulation according to an embodiment of the present application. With reference to FIG. 8, the method provided by the embodiment includes the steps described below.

In step 810, a physiological activity signal of each brain region in at least one brain region of a patient is detected through an implantable electrode device.

In step 820, the detected physiological activity signal of each brain region is compared with a preset detection condition to determine whether the detected physiological activity signal of each brain region belongs to an abnormal physiological activity signal.

In step 830, whether to apply an electrical stimulation signal to the at least one brain region through the implantable electrode device is controlled based on a determination result of the physiological activity signal of each brain region.

In one embodiment, the preset detection condition includes an abnormal state mode. The abnormal state mode includes a change in at least one parameter of an abnormal physiological activity signal of each of the at least one brain region over time.

In one embodiment, the at least one parameter of the abnormal physiological activity signal includes at least one of: an intensity of the abnormal physiological activity signal or a characteristic frequency of the abnormal physiological activity signal.

In one embodiment, the abnormal state mode may be generated based on a preset abnormal state mode.

In one embodiment, the abnormal state mode is obtained by updating the preset abnormal state mode by using at least one historical abnormal physiological activity signal of the patient.

In one embodiment, the abnormal state mode may be generated through training based on a machine learning algorithm.

In one embodiment, the step in which the detected physiological activity signal of each brain region is compared with the preset detection condition includes: performing a similarity comparison between a change in at least one parameter of the detected physiological activity signal of each brain region over time and the change in the at least one parameter of the abnormal physiological activity signal of each brain region over time included in the abnormal state mode.

In one embodiment, the similarity comparison is performed by adopting an autocorrelation algorithm or a cross-correlation algorithm.

In one embodiment, the step in which the physiological activity signal of each brain region in the at least one brain region of the patient is detected through the implantable electrode device includes: periodically detecting the physiological activity signal of the each brain region in the at least one brain region of the patient through the implantable electrode device; or the step in which whether to apply the electrical stimulation signal to the at least one brain region through the implantable electrode device is controlled includes: periodically controlling to apply the electrical stimulation signal to the at least one brain region through the implantable electrode device; or the step in which the physiological activity signal of each brain region in the at least one brain region of the patient is detected through the implantable electrode device includes: periodically detecting the physiological activity signal of each brain region in the at least one brain region of the patient through the implantable electrode device; and the step in which whether to apply the electrical stimulation signal to the at least one brain region through the implantable electrode device is controlled includes: periodically controlling to apply the electrical stimulation signal to the at least one brain region through the implantable electrode device.

In one embodiment, in a case of periodically detecting the physiological activity signal of each brain region in the at least one brain region of the patient through the implantable electrode device and periodically controlling to apply the electrical stimulation signal to the at least one brain region through the implantable electrode device, at a predetermined time interval after the electrical stimulation signal is controlled to be applied to the at least one brain region through the implantable electrode device, the physiological activity signal of each brain region in the at least one brain region of the patient is detected through the implantable electrode device.

In one embodiment, the predetermined time interval is 0.01 milliseconds to 1 hour.

In one embodiment, the step in which whether to apply the electrical stimulation signal to the at least one brain region through the implantable electrode device is controlled includes: controlling to apply the electrical stimulation signal to the at least one brain region for 1 second to 1 hour through the implantable electrode device.

In one embodiment, the step in which whether to apply the electrical stimulation signal to the at least one brain region through the implantable electrode device is controlled includes: controlling the implantable electrode device to apply the electrical stimulation signal to the at least one brain region and controlling at least one of: a duration for applying the applied electrical stimulation signal or an amplitude of the applied electrical stimulation signal.

In one embodiment, the duration and amplitude of the applied electrical stimulation signal are positively correlated with the detected physiological activity signal.

In one embodiment, the step in which whether to apply the electrical stimulation signal to the at least one brain region through the implantable electrode device is controlled includes: intermittently applying the electrical stimulation signal to the at least one brain region through the implantable electrode device.

In one embodiment, a duration, an amplitude, and a frequency of the intermittently applied electrical stimulation signal are positively correlated with the detected physiological activity signal.

In one embodiment, an amplitude range of the electrical stimulation signal is 0.5 V to 10V.

In one embodiment, the step in which the physiological activity signal of each brain region in the at least one brain region of the patient is detected through the implantable electrode device includes: detecting the physiological activity signal of each brain region in the at least one brain region of the patient through at least one detection contact of the implantable electrode device; and the step in which whether to apply the electrical stimulation signal to the at least one brain region through the implantable electrode device is controlled includes: controlling whether to apply the electrical stimulation signal to the at least one brain region through at least one stimulation contact of the implantable electrode device.

In one embodiment, the at least one detection contact includes multiple detection contacts, and the at least one stimulation contact includes multiple stimulation contacts.

In one embodiment, the number of the detection contacts is the same as the number of the stimulation contacts, and the range of a brain region detected by each detection contact does not overlap the range of a brain region affected by a respective stimulation contact corresponding to the each detection contact.

In one embodiment, the number of the detection contacts is the same as the number of the stimulation contacts, and the range of a brain region detected by each detection contact does not overlap the range of a brain region affected by a respective stimulation contact corresponding to the each detection contact.

In one embodiment, the at least one brain region includes at least one of: a cerebral cortex region or a deep brain region.

In one embodiment, the step in which the physiological activity signal of each brain region in the at least one brain region of the patient is detected through the implantable electrode device includes: detecting the physiological activity signal of each brain region in the at least one of the cerebral cortex region or the deep brain region of the patient through the multiple detection contacts implanted in the at least one of: the cerebral cortex region or the deep brain region.

In one embodiment, the at least one brain region includes at least one brain region of a left brain region and at least one brain region of a right brain region.

In one embodiment, the at least one brain region includes at least two brain regions of a left brain region and/or at least two brain regions of a right brain region.

In one embodiment, the step in which whether to apply the electrical stimulation signal to the at least one brain region through the implantable electrode device is controlled based on the determination result includes: controlling whether to apply the electrical stimulation signal to the deep brain region through the multiple stimulation contacts implanted in the deep brain region based on the determination result.

In one embodiment, the deep brain region includes at least one of: nucleus accumbens or anterior limb of internal capsule.

In one embodiment, the deep brain region includes at least one of nucleus accumbens or anterior limb of internal capsule of a left deep brain region and at least one of nucleus accumbens or anterior limb of internal capsule of a right deep brain region.

In one embodiment, the deep brain region includes at least one of: nucleus accumbens, anterior limb of internal capsule, ventral capsule/ventral striatum, anterior thalamic radiation, medial forebrain bundle, bed nucleus of stria terminalis, subgenual cingulate cortex, inferior thalamic peduncle, amygdala, anterior cingulated cortex, lateral habenula, hippocampus, subthalamic nucleus, or globus pallidus pars interna.

In one embodiment, the cerebral cortex region includes at least one of: prefrontal cortex, orbitofrontal cortex, parietal cortex, or temporal cortex.

In one embodiment, the prefrontal cortex includes at least one of: dorsomedial prefrontal cortex or dorsolateral prefrontal cortex.

In one embodiment, the at least one brain region includes: ventral capsule/ventral striatum, bed nucleus of stria terminalis, anterior limb of internal capsule, and nucleus accumbens.

In one embodiment, the at least one brain region includes anterior limb of internal capsule and cingulated cortex.

In one embodiment, the step in which whether to apply the electrical stimulation signal to the at least one brain region through the implantable electrode device is controlled includes: through the implantable electrode device, applying an electrical stimulation signal with a voltage range of 2.5 V to 8 V, a pulse width range of 120 μs to 210 μs, and a frequency range of 90 Hz to 135 Hz to the ventral capsule/ventral striatum, applying an electrical stimulation signal with a voltage range of 3 V to 10.5 V, a pulse width range of 90 μs to 450 μs, and a frequency range of 85 Hz to 135 Hz to the bed nucleus of stria terminalis, applying an electrical stimulation signal with a voltage range of 2 V to 7 V, a pulse width range of 90 μs to 300 μs, and a frequency range of 130 Hz to 185 Hz to the anterior limb of internal capsule, and applying an electrical stimulation signal with a voltage range of 4 V to 7 V, a pulse width range of 90 μs to 240 μs, and a frequency range of 100 Hz to 150 Hz to the nucleus accumbens.

In one embodiment, the step in which whether to apply the electrical stimulation signal to the at least one brain region through the implantable electrode device is controlled includes: through the implantable electrode device, applying an electrical stimulation signal with a voltage range of 2.5 V to 6 V, a pulse width range of 90 μs to 300 μs, and a frequency range of 130 Hz to 180 Hz to the anterior limb of internal capsule, and applying an electrical stimulation signal with a voltage range of 2 V to 10 V, a pulse width range of 90 μs to 300 μs, and a frequency range of 130 Hz to 210 Hz to the cingulated cortex.

In one embodiment, the step in which the physiological activity signal of each brain region in the at least one brain region of the patient is detected through the implantable electrode device includes: detecting the physiological activity signal of each brain region in the at least one brain region of the patient through multiple groups of implantable electrodes of the implantable electrode device; and the step in which whether to apply the electrical stimulation signal to the at least one brain region through the implantable electrode device is controlled based on the determination result of the physiological activity signal of each brain region includes: controlling whether to apply the electrical stimulation signal to the at least one brain region through the multiple groups of implantable electrodes based on the determination result of the physiological activity signal of each brain region, where at least one group of implantable electrodes includes both a detection contact and a stimulation contact.

In one embodiment, the step in which the physiological activity signal of each brain region in the at least one brain region of the patient is detected through the implantable electrode device includes: detecting the physiological activity signal of each brain region in the at least one brain region of the patient through at least one detection contact of the implantable electrode device, where the diameter range of each of the at least one detection contact is 0.1 mm to 3 mm, an amplitude range of a physiological activity signal acquired by each of the at least one detection contact is 5 uV to 12.5 mV, and a frequency range of the physiological activity signal is 0.5 Hz to 150 Hz.

In one embodiment, the step in which the physiological activity signal of each brain region in the at least one brain region of the patient is detected through the implantable electrode device includes: detecting the physiological activity signal of the each brain region in the at least one brain region of the patient through at least one detection contact of the implantable electrode device, wherein a diameter range of each of the at least one detection contact is 0.1 mm to 0.5 mm, an amplitude range of a physiological activity signal acquired by each of the at least one detection contact is 5 uV to 10 mV, and a frequency range of the physiological activity signal is 150 Hz to 30000 Hz.

In one embodiment, the step in which the physiological activity signal of each brain region in the at least one brain region of the patient is detected through the implantable electrode device includes: detecting the physiological activity signal of each brain region in the at least one brain region of the patient through at least one detection contact of the implantable electrode device, where the diameter range of each of the at least one detection contact is 5 um to 100 um.

In one embodiment, the electrical stimulation signal is used for the treatment of mental and behavioral disorders.

In one embodiment, the mental and behavioral disorders includes addiction, obsessive compulsive disorders, depressive disorders, anxiety disorders, schizophrenia, anorexia nervosa, Tourette's disorder, or Autism disorder.

In one embodiment, the electrical stimulation signal is used for the treatment of addition, and the addition includes at least one of: substance addiction or non-substance addiction.

In one embodiment, the substance addiction includes drug addiction, alcohol addiction, nicotine addiction, caffeine addiction, and the non-substance addiction includes gambling addiction, sexual behavior disorder/addiction, or gaming disorder.

In one embodiment, the drug addiction comprises legal drug addiction or illegal drug addiction; the legal drug addiction comprises hallucinogen addiction, inhalant drug addiction, anesthetic drug addiction, sedative drug addiction, hypnotic drug addiction, anxiolytic drug addiction, or stimulant drug addiction; the illegal drug addiction includes at least one of: opioid drug addiction, cannabis addiction, methamphetamine addiction, or LSD addiction.

In one embodiment, the electrical stimulation signal is used for the treatment of drug addiction, obsessive compulsive disorders, or depressive disorders.

In one embodiment, the electrical stimulation signal is used for the treatment of drug addiction, and the electrical stimulation signal inhibits drug addition of the patient.

In one embodiment, the electrical stimulation signal is used for the treatment of obsessive compulsive disorders.

In one embodiment, the electrical stimulation signal is used for the treatment of depressive disorders.

In one embodiment, the step in which whether to apply the electrical stimulation signal to the at least one brain region through the implantable electrode device is controlled includes: controlling whether to apply the electrical stimulation signal to the at least one brain region through multiple stimulation contacts of the implantable electrode device, where the multiple stimulation contacts apply the same electrical stimulation signal.

In one embodiment, the step in which whether to apply the electrical stimulation signal to the at least one brain region through the implantable electrode device is controlled includes: controlling whether to apply the electrical stimulation signal to the at least one brain region through multiple stimulation contacts of the implantable electrode device, where the multiple stimulation contacts apply different electrical stimulation signals.

In one embodiment, the step in which whether to apply the electrical stimulation signal to the at least one brain region through the implantable electrode device is controlled includes: controlling whether to apply the electrical stimulation signal to the at least one brain region through multiple stimulation contacts of the implantable electrode device, where at least one parameter of the electrical stimulation signal applied by the multiple stimulation contacts is associated with each other.

In one embodiment, the step in which whether to apply the electrical stimulation signal to the at least one brain region through the implantable electrode device is controlled includes: controlling whether to apply the electrical stimulation signal to the at least one brain region through multiple stimulation contacts of the implantable electrode device, where each stimulation contact corresponds to at least one detection contact, the each stimulation contact and the at least one detection contact corresponding to the each stimulation contact constitute a detection-stimulation group, and each detection-stimulation group independently detects the physiological activity signal and applies the electrical stimulation signal.

In one embodiment, the time when each detection-stimulation group performs physiological activity signal detection is not synchronized with the time when at least one detection-stimulation group performs physiological activity signal detection, and the time when the each detection-stimulation group applies the electrical stimulation signal is not synchronized with the time when the at least one detection-stimulation group applies the electrical stimulation signal.

In one embodiment, different detection-stimulation groups perform physiological activity signal detection synchronously, and different detection-stimulation groups perform electrical stimulation signal application synchronously.

FIG. 9 illustrates a structural diagram of a device for neural stimulation according to another embodiment of the present application. The device for neuro-stimulation includes a non-transitory storage medium 910, a processor 920, and an implantable electrode device 930. The non-transitory storage medium 910 has an instruction executable by the processor, and the instruction is run to perform the method provided by any embodiment of the present application.

It is to be noted that although various modules or sub-modules of the device for nerve stimulation are mentioned in the above description, such a division is only exemplary and not mandatory. In fact, according to the embodiments of the present application, features and functions of two or more modules described above may be embodied in one module. The features and functions of one module described above may be embodied by multiple modules.

Those of ordinary skill in the art may understand and implement other changes to the disclosed embodiments by studying specification, the contents of the disclosure, the drawings, and appended claims. In the claims, the word “comprise” does not exclude other elements or steps and the word “one” and “each” does not exclude the plural. In practical application of the present application, a component may perform functions of multiple technical features cited in the claims. Any reference numeral in the claims shall not be construed as limiting the scope of the present application.

What is claimed is:

1. A method for neuro-stimulation, comprising:
detecting a physiological activity signal of each brain region in at least one brain region of a patient through an implantable electrode device;
comparing the detected physiological activity signal of the each brain region with a preset detection condition to determine whether the detected physiological activity signal of the each brain region belongs to an abnormal physiological activity signal; and
controlling whether to apply an electrical stimulation signal to the at least one brain region through the implantable electrode device based on a determination result of the physiological activity signal of each brain region.

2. The method of claim 1, wherein the preset detection condition comprises an abnormal state mode, wherein the abnormal state mode comprises a change in at least one parameter of an abnormal physiological activity signal of each of the at least one brain region over time.

3. The method of claim 2, wherein the at least one parameter of the abnormal physiological activity signal comprises at least one of an intensity of the abnormal physiological activity signal or a characteristic frequency of the abnormal physiological activity signal.

4. The method of claim 2, wherein the abnormal state mode is generated based on a preset abnormal state mode.

5. The method of claim 4, wherein the abnormal state mode is obtained by updating the preset abnormal state mode by using at least one historical abnormal physiological activity signal of the patient.

6. The method of claim 5, wherein the abnormal state mode is generated through training based on a machine learning algorithm.

7. The method of claim 2, wherein comparing the detected physiological activity signal of the each brain region with the preset detection condition comprises:
performing a similarity comparison between a change in at least one parameter of the detected physiological activity signal of the each brain region over time and the change in the at least one parameter of the abnormal physiological activity signal of the each brain region over time comprised in the abnormal state mode.

8. The method of claim 7, wherein the similarity comparison is performed by adopting an autocorrelation algorithm or a cross-correlation algorithm.

9. The method of claim 1, wherein
detecting the physiological activity signal of the each brain region in the at least one brain region of the patient through the implantable electrode device comprises: periodically detecting the physiological activity signal of the each brain region in the at least one brain region of the patient through the implantable electrode device; or
controlling whether to apply the electrical stimulation signal to the at least one brain region through the implantable electrode device comprises: periodically controlling to apply the electrical stimulation signal to the at least one brain region through the implantable electrode device; or
detecting the physiological activity signal of the each brain region in the at least one brain region of the patient through the implantable electrode device comprises: periodically detecting the physiological activity signal of the each brain region in the at least one brain region of the patient through the implantable electrode device; and controlling whether to apply the electrical stimulation signal to the at least one brain region through the implantable electrode device comprises: periodically controlling to apply the electrical stimulation signal to the at least one brain region through the implantable electrode device.

10. The method of claim 9, further comprising: in a case of periodically detecting the physiological activity signal of the each brain region in the at least one brain region of the patient through the implantable electrode device and periodically controlling to apply the electrical stimulation signal to the at least one brain region through the implantable electrode device, at a predetermined time interval after controlling to apply the electrical stimulation signal to the at least one brain region through the implantable electrode device, executing the step of detecting the physiological activity signal of the each brain region in the at least one brain region of the patient through the implantable electrode device.

11. The method of claim 10, wherein the predetermined time interval is 0.01 milliseconds to 1 hour.

12. The method of claim 1, wherein controlling whether to apply the electrical stimulation signal to the at least one brain region through the implantable electrode device comprises:
controlling to apply the electrical stimulation signal to the at least one brain region for 1 second to 1 hour through the implantable electrode device.

13. The method of claim 1, wherein controlling whether to apply the electrical stimulation signal to the at least one brain region through the implantable electrode device comprises:
controlling the implantable electrode device to apply the electrical stimulation signal to the at least one brain region and controlling at least one of a duration for applying the applied electrical stimulation signal or an amplitude of the applied electrical stimulation signal.

14. The method of claim 13, wherein the duration and amplitude of the applied electrical stimulation signal are positively correlated with the detected physiological activity signal.

15. The method of claim 1, wherein controlling whether to apply the electrical stimulation signal to the at least one brain region through the implantable electrode device comprises:
intermittently applying the electrical stimulation signal to the at least one brain region through the implantable electrode device.

16. The method of claim 15, wherein a duration, an amplitude, and a frequency of the intermittently applied electrical stimulation signal are positively correlated with the detected physiological activity signal.

17. The method of claim 1, wherein an amplitude range of the electrical stimulation signal is 0.5 V to 10 V.

18. The method of claim 1, wherein detecting the physiological activity signal of the each brain region in the at least one brain region of the patient through the implantable electrode device comprises: detecting the physiological activity signal of the each brain region in the at least one brain region of the patient through at least one detection contact of the implantable electrode device; and
controlling whether to apply the electrical stimulation signal to the at least one brain region through the implantable electrode device comprises: controlling whether to apply the electrical stimulation signal to the at least one brain region through at least one stimulation contact of the implantable electrode device.

19. The method of claim 18, wherein the at least one detection contact comprises a plurality of detection contacts, and the at least one stimulation contact comprises a plurality of stimulation contacts.

20. The method of claim 19, wherein a number of the plurality of detection contacts is the same as a number of the plurality of stimulation contacts, and a range of a brain region detected by each of the plurality of detection contacts overlaps a range of a brain region affected by a respective one of the plurality of stimulation contacts corresponding to the each of the plurality of detection contacts.

21. The method of claim 19, wherein a number of the plurality of detection contacts is the same as a number of the plurality of stimulation contacts, and a range of a brain region detected by each of the plurality of detection contacts does not overlap a range of a brain region affected by a respective one of the plurality of stimulation contacts corresponding to the each of the plurality of detection contacts.

22. The method of claim 20, wherein the at least one brain region comprises at least one of a cerebral cortex region or a deep brain region.

23. The method of claim 22, wherein detecting the physiological activity signal of the each brain region in the at least one brain region of the patient through the implantable electrode device comprises: detecting the physiological activity signal of the each brain region in the at least one of the cerebral cortex region or the deep brain region of the patient through the plurality of detection contacts implanted in the at least one of the cerebral cortex region or the deep brain region.

24. The method of claim 1, wherein the at least one brain region comprises at least one brain region of a left brain region and at least one brain region of a right brain region.

25. The method of claim 1, wherein the at least one brain region comprises at least one of: at least two brain regions of a left brain region or at least two brain regions of a right brain region.

26. The method of claim 22, wherein controlling whether to apply the electrical stimulation signal to the at least one brain region through the implantable electrode device based on the determination result comprises:
controlling whether to apply the electrical stimulation signal to the deep brain region through the plurality of stimulation contacts implanted in the deep brain region based on the determination result.

27. The method of claim 22, wherein the deep brain region comprises at least one of: nucleus accumbens or anterior limb of internal capsule.

28. The method of claim 27, wherein the deep brain region comprises at least one of nucleus accumbens or anterior limb of internal capsule of a left deep brain region and at least one of nucleus accumbens or anterior limb of internal capsule of a right deep brain region.

29. The method of claim 22, wherein the deep brain region comprises at least one of: nucleus accumbens, anterior limb of internal capsule, ventral capsule/ventral striatum, anterior thalamic radiation, medial forebrain bundle, bed nucleus of stria terminalis, subgenual cingulate cortex, inferior thalamic peduncle, amygdala, anterior cingulated cortex, lateral habenula, hippocampus, subthalamic nucleus, or globus pallidus pars interna.

30. The method of claim 29, wherein the cerebral cortex region comprises at least one of:
prefrontal cortex, orbitofrontal cortex, parietal cortex, or temporal cortex.

31. The method of claim 30, wherein the prefrontal cortex comprises at least one of dorsomedial prefrontal cortex or dorsolateral prefrontal cortex.

32. The method of claim 1, wherein the at least one brain region comprises ventral capsule/ventral striatum, bed nucleus of stria terminalis, anterior limb of internal capsule, and nucleus accumbens.

33. The method of claim 1, wherein the at least one brain region comprises: anterior limb of internal capsule and cingulated cortex.

34. The method of claim 32, wherein controlling whether to apply the electrical stimulation signal to the at least one brain region through the implantable electrode device comprises:
through the implantable electrode device, applying an electrical stimulation signal with a voltage range of 2.5 V to 8 V, a pulse width range of 120 μs to 210 μs, and a frequency range of 90 Hz to 135 Hz to the ventral capsule/ventral striatum, applying an electrical stimulation signal with a voltage range of 3 V to 10.5 V, a pulse width range of 90 μs to 450 μs, and a frequency range of 85 Hz to 135 Hz to the bed nucleus of stria terminalis, applying an electrical stimulation signal with a voltage range of 2 V to 7 V, a pulse width range of 90 μs to 300 μs, and a frequency range of 130 Hz to 185 Hz to the anterior limb of internal capsule, and applying an electrical stimulation signal with a voltage range of 4 V to 7 V, a pulse width range of 90 μs to 240 μs, and a frequency range of 100 Hz to 150 Hz to the nucleus accumbens.

35. The method of claim 33, wherein controlling whether to apply the electrical stimulation signal to the at least one brain region through the implantable electrode device comprises:
through the implantable electrode device, applying an electrical stimulation signal with a voltage range of 2.5 V to 6 V, a pulse width range of 90 μs to 300 μs, and a frequency range of 130 Hz to 180 Hz to the anterior limb of internal capsule, and applying an electrical stimulation signal with a voltage range of 2 V to 10 V, a pulse width range of 90 us to 300 μs, and a frequency range of 130 Hz to 210 Hz to the cingulated cortex.

36. The method of claim 1, wherein detecting the physiological activity signal of the each brain region in the at least one brain region of the patient through the implantable electrode device comprises: detecting the physiological activity signal of the each brain region in the at least one brain region of the patient through a plurality of groups of implantable electrodes of the implantable electrode device, and controlling whether to apply the electrical stimulation signal to the at least one brain region through the implantable electrode device based on the determination result of the physiological activity signal of the each brain region comprises: controlling whether to apply the electrical stimulation signal to the at least one brain region through the plurality of groups of implantable electrodes based on the determination result of the physiological activity signal of the each brain region, wherein at least one group of implantable electrodes comprises a detection contact and a stimulation contact.

37. The method of claim 1, wherein detecting the physiological activity signal of the each brain region in the at least one brain region of the patient through the implantable electrode device comprises:
detecting the physiological activity signal of the each brain region in the at least one brain region of the patient through at least one detection contact of the implantable electrode device, wherein a diameter range of each of the at least one detection contact is 0.1 mm to 3 mm, an amplitude range of a physiological activity signal acquired by each of the at least one detection contact is 5 uV to 12.5 mV, and a frequency range of the physiological activity signal is 0.5 Hz to 150 Hz.

38. The method of claim 1, wherein detecting the physiological activity signal of the each brain region in the at least one brain region of the patient through the implantable electrode device comprises:
detecting the physiological activity signal of the each brain region in the at least one brain region of the patient through at least one detection contact of the implantable electrode device, wherein a diameter range of each of the at least one detection contact is 0.1 mm to 0.5 mm, an amplitude range of a physiological activity signal acquired by each of the at least one detection contact is 5 uV to 10 mV, and a frequency range of the physiological activity signal is 150 Hz to 30000 Hz.

39. The method of claim 1, wherein detecting the physiological activity signal of the each brain region in the at least one brain region of the patient through the implantable electrode device comprises:
detecting the physiological activity signal of the each brain region in the at least one brain region of the patient through at least one detection contact of the implantable electrode device, wherein a diameter range of each of the at least one detection contact is 5 um to 100 um.

40. The method of claim 1, wherein the electrical stimulation signal is used for the treatment of mental and behavioral disorders.

41. The method of claim 40, wherein the mental and behavioral disorders comprises addiction, obsessive compulsive disorders, depressive disorders, anxiety disorders, schizophrenia, anorexia nervosa, Tourette's disorder, or Autism disorder.

42. The method of claim 41, wherein the addiction comprises substance addiction or non-substance addiction.

43. The method of claim 42, wherein the substance addiction comprises drug addiction, alcohol addiction, nicotine addiction, or caffeine addiction, and the non-substance addiction comprises gambling addiction, sexual behavior disorder/addiction, or gaming disorder.

44. The method of claim 43, wherein the drug addiction comprises legal drug addiction or illegal drug addiction;
the legal drug addiction comprises hallucinogen addiction, inhalant drug addiction, anesthetic drug addiction, sedative drug addiction, hypnotic drug addiction, anxiolytic drug addiction, or stimulant drug addiction;
the illegal drug addiction comprises: opioid drug addiction, cannabis addiction,
methamphetamine addiction, or lysergic acid diethylamide (LSD) addiction.

45. The method of claim 1, wherein the electrical stimulation signal is used for the treatment of drug addiction, obsessive compulsive disorders, or depressive disorders.

46. The method of claim 45, wherein the electrical stimulation signal is used for the treatment of drug addiction, and the electrical stimulation signal inhibits drug addition of the patient.

47. The method of claim 34, wherein the electrical stimulation signal is used for the treatment of obsessive compulsive disorders.

48. The method of claim 35, wherein the electrical stimulation signal is used for the treatment of depressive disorders.

49. The method of claim 1, wherein controlling whether to apply the electrical stimulation signal to the at least one brain region through the implantable electrode device comprises:
controlling whether to apply the electrical stimulation signal to the at least one brain region through a plurality of stimulation contacts of the implantable electrode device, wherein the plurality of stimulation contacts apply a same electrical stimulation signal.

50. The method of claim 1, wherein controlling whether to apply the electrical stimulation signal to the at least one brain region through the implantable electrode device comprises:
controlling whether to apply the electrical stimulation signal to the at least one brain region through a plurality of stimulation contacts of the implantable electrode device, wherein the plurality of stimulation contacts apply different electrical stimulation signals.

51. The method of claim 1, wherein controlling whether to apply the electrical stimulation signal to the at least one brain region through the implantable electrode device comprises: controlling whether to apply the electrical stimulation signal to the at least one brain region through a plurality of stimulation contacts of the implantable electrode device, wherein at least one parameter of the electrical stimulation signal applied by the plurality of stimulation contacts is associated with each other.

52. The method of claim 1, wherein controlling whether to apply the electrical stimulation signal to the at least one brain region through the implantable electrode device comprises: controlling whether to apply the electrical stimulation signal to the at least one brain region through a plurality of stimulation contacts of the implantable electrode device, wherein each of the plurality of stimulation contacts corresponds to at least one detection contact, the each of the plurality of stimulation contacts and the at least one detection contact corresponding to the each of the plurality of stimulation contacts constitute a detection-stimulation group, and each detection-stimulation group independently detects the physiological activity signal and applies the electrical stimulation signal.

53. The method of claim 52, wherein a time when each detection-stimulation group performs physiological activity signal detection is not synchronized with a time when at least one detection-stimulation group performs physiological activity signal detection, and a time when the each detection-stimulation group applies the electrical stimulation signal is not synchronized with a time when the at least one detection-stimulation group applies the electrical stimulation signal.

54. The method of claim 52, wherein different detection-stimulation groups perform physiological activity signal detection synchronously, and different detection-stimulation groups perform electrical stimulation signal application synchronously.

55. A device for neuro-stimulation, comprising:
an implantable electrode device, which is configured to detect a physiological activity signal of each brain region in at least one brain region of a patient; and
a controller, which is connected to the implantable electrode device and configured to acquire the physiological activity signal of the each brain region detected by the implantable electrode device, and compare the physiological activity signal of the each brain region with a preset detection condition to determine whether the detected physiological activity signal of the each brain region belongs to an abnormal physiological activity signal; and control whether to apply an electrical stimulation signal to the at least one brain region through the implantable electrode device based on a determination result of the physiological activity signal of each brain region.

56. The device of claim 55, wherein the implantable electrode device comprises a first implantable electrode and a second implantable electrode;
wherein the first implantable electrode is configured to be implanted into a first brain region of the patient and detect a physiological activity signal of the first brain region; and
the second implantable electrode is configured to be implanted into a second brain region of the patient and detect a physiological activity signal of the second brain region.

57. The device of claim 56, wherein the controller is configured to control whether to apply the electrical stimulation signal to the at least one brain region through the implantable electrode device in the following manner: controlling the first implantable electrode and the second implantable electrode to synchronously or asynchronously apply the electrical stimulation signal to the first brain region and the second brain region.

58. The device of claim 57, wherein the electrical stimulation signal applied to the first brain region and the electrical stimulation signal applied to the second brain region differ from each other in at least one of parameters: a duration, an amplitude, a frequency, or a pulse width.

59. The device of claim 55, wherein the preset detection condition comprises an abnormal state mode, wherein the abnormal state mode comprises a change in at least one parameter of an abnormal physiological activity signal of each of the at least one brain region over time.

60. The device of claim 59, wherein the at least one parameter of the abnormal physiological activity signal comprises at least one of an intensity of the abnormal physiological activity signal or a characteristic frequency of the abnormal physiological activity signal.

61. The device of claim 58, wherein the abnormal state mode is generated based on a preset abnormal state mode.

62. The device of claim 61, wherein the abnormal state mode is obtained by updating the preset abnormal state mode by using at least one historical abnormal physiological activity signal of the patient.

63. The device of claim 62, wherein the abnormal state mode is generated through training based on a machine learning algorithm.

64. The device of claim 59, wherein the controller is configured to compare the physiological activity signal of the each brain region with the preset detection condition in the following manner:
performing a similarity comparison between a change in at least one parameter of the detected physiological activity signal of the each brain region over time and the change in the at least one parameter of the abnormal physiological activity signal of the each brain region over time comprised in the abnormal state mode.

65. The device of claim 64, wherein the similarity comparison is performed by adopting an autocorrelation algorithm or a cross-correlation algorithm.

66. A device for neuro-stimulation, comprising a non-transitory storage medium, a processor, and an implantable electrode device, wherein the non-transitory storage medium has an instruction executable by the processor, and the instruction is run to perform the following steps:
detecting a physiological activity signal of each brain region in at least one brain region of a patient through the implantable electrode device;
comparing the detected physiological activity signal of the each brain region with a preset detection condition to determine whether the detected physiological activity signal of the each brain region belongs to an abnormal physiological activity signal; and
controlling whether to apply an electrical stimulation signal to the at least one brain region through the implantable electrode device based on a determination result of the physiological activity signal of each brain region.

67. The device of claim 66, wherein the preset detection condition comprises an abnormal state mode, wherein the abnormal state mode comprises a change in at least one parameter of an abnormal physiological activity signal of the each of the at least one brain region over time.

68. The device of claim 67, wherein the at least one parameter of the abnormal physiological activity signal comprises at least one of an intensity of the abnormal physiological activity signal or a characteristic frequency of the abnormal physiological activity signal.

69. The device of claim 67, wherein the abnormal state mode is generated based on a preset abnormal state mode.

70. The device of claim 69, wherein the abnormal state mode is obtained by updating the preset abnormal state mode by using at least one historical abnormal physiological activity signal of the patient.

71. The device of claim 67, wherein comparing the detected physiological activity signal of each brain region with the preset detection condition comprises:
performing a similarity comparison between a change in at least one parameter of the detected physiological activity signal of the each brain region over time and the change in the at least one parameter of the abnormal physiological activity signal of the each brain region over time comprised in the abnormal state mode.

72. The device of claim 66, wherein
detecting the physiological activity signal of the each brain region in the at least one brain region of the patient through the implantable electrode device comprises: periodically detecting the physiological activity signal of the each brain region in the at least one brain region of the patient through the implantable electrode device; or
controlling whether to apply the electrical stimulation signal to the at least one brain region through the implantable electrode device comprises: periodically controlling to apply the electrical stimulation signal to the at least one brain region through the implantable electrode device; or
detecting the physiological activity signal of the each brain region in the at least one brain region of the patient through the implantable electrode device comprises: periodically detecting the physiological activity signal of the each brain region in the at least one brain region of the patient through the implantable electrode device; and controlling whether to apply the electrical stimulation signal to the at least one brain region through the implantable electrode device comprises: periodically controlling to apply the electrical stimulation signal to the at least one brain region through the implantable electrode device.

73. The device of claim 66, wherein controlling whether to apply the electrical stimulation signal to the at least one brain region through the implantable electrode device comprises: controlling the implantable electrode device to apply the electrical stimulation signal to the at least one brain region and controlling at least one of a duration for applying the electrical stimulation signal or an amplitude of the electrical stimulation signal.

74. The device of claim 66, wherein controlling whether to apply the electrical stimulation signal to the at least one brain region through the implantable electrode device comprises:
intermittently applying the electrical stimulation signal to the at least one brain region through the implantable electrode device.

75. The device of claim 66, wherein the implantable electrode device comprises a plurality of detection contacts for detecting the physiological activity signal of the each brain region in the at least one brain region, and a plurality of stimulation contacts for applying the electrical stimulation signal to the at least one brain region.

76. The device of claim 75, wherein a number of the plurality of detection contacts is the same as a number of the plurality of stimulation contacts, and a range of a brain region detected by each of the plurality of detection contacts overlaps a range of a brain region affected by a respective one of the plurality of stimulation contacts corresponding to the each of the plurality of detection contacts.

77. The device of claim 76, wherein the plurality of detection contacts and the plurality of detection contacts of the implantable electrode device are implanted into at least one of a cerebral cortex region or a deep brain region.

78. The device of claim 77, wherein the deep brain region comprises at least one of: nucleus accumbens or anterior limb of internal capsule.

79. The device of claim 78, wherein the deep brain region comprises at least one of nucleus accumbens or anterior limb of internal capsule of a left deep brain region and at least one of nucleus accumbens or anterior limb of internal capsule of a right deep brain region.

80. The device of claim 66, wherein the implantable electrode device comprises a plurality of stimulation contacts for applying the electrical stimulation signal to the at least one brain region, and at least one parameter of the electrical stimulation signal applied by the plurality of stimulation contacts is associated with each other.

81. The device of claim 66, wherein the implantable electrode device comprises a plurality of stimulation contacts for applying the electrical stimulation signal to the at least one brain region, each of the plurality of stimulation contacts corresponds to at least one detection contact, the each of the plurality of stimulation contacts and the at least one detection contact corresponding to the each of the plurality of stimulation contacts constitute a detection-stimulation group, and each detection-stimulation group independently detects the physiological activity signal and applies the electrical stimulation signal.

82. A computer-readable storage medium storing a computer program, wherein a processor executes the program to perform the method for neuro-stimulation of claim 1.
